(19) United States
(12) Patent Application Publication
(10) Pub. No.: US 2026/0011433 A1
(43) Pub. Date: Jan. 8, 2026

(54) AUTOMATIC MEDICAL CODING DETERMINATION

(71) Applicant: AI Healthcare, LLC, Salida, CO (US)

(72) Inventors: Paul Eugene Bergmann, Shoreview, MN (US); Mariana Rodrigues Lourenço, Coimbra (PT); Rui Pedro Pereira Mendes, Coimbra (PT)

(73) Assignee: AI Healthcare, LLC, Salida, CO (US)

(21) Appl. No.: 18/765,093

(22) Filed: Jul. 5, 2024

(51) Int. Cl. G16H 40/20

(52) U.S. Cl. CPC G16H 40/20

(57) ABSTRACT

Automatically determining medical codes based on medical documents may be provided. Automatically determining medical codes can comprise processing and extracting data from a medical document. One or more medical codes can be determined for the medical document using a multi-tier plurality of models. The multi-tier plurality of models can comprise one or more first-level medical models for determining a medical code category, and a plurality of second-level medical models. One or more second-level models may be selected based on determined medical code categories, and the second-level models may be applied to determine medical codes. Confidence scores can be assigned to the determined medical codes, and a medical coding report can be generated comprising the one or more medical codes and the confidence scores.

### TECHNICAL FIELD

The present disclosure relates generally to automatically determining medical codes based on medical documents.

### BACKGROUND

Medical codes, such as medical procedure codes, such as Current Procedural Terminology (CPT) codes, and medical classification codes, such as International Classification of Diseases, 10th Revision (ICD-10) codes, are essential elements in the healthcare system, serving distinct but complementary functions. Medical procedure codes such as CPT codes describe medical, surgical, and diagnostic services, allowing healthcare providers to communicate uniform information about these procedures. Medical classification codes such as ICD-10, on the other hand, provide a standardized system for classifying diseases and health conditions. ICD-10 codes are used globally for epidemiological, health management, and clinical purposes. While medical procedure codes such as CPT codes focus on describing medical procedures and services within the United States, medical classification codes such as ICD-10 codes focus on diseases and health conditions, ensuring consistency in diagnosis and treatment information across different regions and healthcare systems. Together, these coding systems can facilitate accurate and standardized communication of medical information, crucial for treatment, billing, and statistical analysis in healthcare.

Currently, medical coding processes are time-consuming and labor-intensive, requiring medical coders to review clinical documentation and assign appropriate codes manually. This manual approach is not only expensive and prone to errors but also subject to non-uniform practices and potential data security issues. These challenges emphasize the need for improved medical coding processes that enhance efficiency, accuracy, and security.

### SUMMARY

Systems and methods for automatically determining medical codes based on medical documents may be provided. Automatically determining medical codes can comprise processing and extracting data from a medical document. One or more medical codes can be determined for the medical document using a multi-tier plurality of models. The multi-tier plurality of models can comprise one or more first-level medical models for determining a medical code category, and a plurality of second-level medical models. One or more second-level models may be selected based on determined medical code categories, and the second-level models may be applied to determine medical codes. Confidence scores can be assigned to the determined medical codes, and a medical coding report can be generated comprising the one or more medical codes and the confidence scores.

### BRIEF DESCRIPTION OF THE DRAWINGS

The accompanying drawings, which are incorporated in and constitute a part of this disclosure, illustrate various embodiments of the present disclosure. In the drawings:

FIG. 1 is a block diagram of an operating environment for automatically determining medical codes in accordance with aspects of the present disclosure.

FIG. 2 is a diagram of finalized medical coding including a medical coding report generated based on a medical document in accordance with aspects of the present disclosure.

FIG. 3 is a block diagram of a multi-tier set of models for determining medical codes in accordance with aspects of the present disclosure.

FIG. 4 is a flow chart of a method for selecting a model for determining medical codes in accordance with aspects of the present disclosure.

FIG. 5 is a diagram of a deployment of models for determining medical codes in accordance with aspects of the present disclosure.

FIG. 6 is a block diagram of a model set for determining evaluation and management medical codes in accordance with aspects of the present disclosure.

FIG. 7 is a flow chart of a method for training models for determining medical codes in accordance with aspects of the present disclosure.

FIG. 8 is a flow chart of a method for automatically determining medical codes based on medical documents in accordance with aspects of the present disclosure.

FIG. 9 is a flow chart of a method for automatically determining medical codes in accordance with aspects of the present disclosure.

FIG. 10 is a block diagram of a computing device in accordance with aspects of the present disclosure.

### DETAILED DESCRIPTION

Both the foregoing summary and the following detailed description are examples and explanatory only and should not be considered to restrict the disclosure's scope, as described, and claimed. Furthermore, features and/or variations may be provided in addition to those described. For example, embodiments of the disclosure may be directed to various feature combinations and sub-combinations described in the example embodiments.

The following detailed description refers to the accompanying drawings. Wherever possible, the same reference numbers are used in the drawings and the following description to refer to the same or similar elements. While embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims.

Methods and systems described herein can utilize a multi-tier set of models, such as a two-tier set of models, to determine medical procedure codes, such as CPT codes, and medical classification codes, such as ICD-10 codes, based on medical documents. In general, a medical coding system can perform the coding in three parts: an input stage, a processing stage, and an output stage. During the input stage, the system receives a medical document, such as a medical note. The system prepares and extracts data (e.g., text) from the document, such as a narration of a procedure that was performed. To do so, the system may perform optical character recognition (OCR), natural language processing (NLP), or other image or text processing tasks to extract the relevant text.

During the processing stage, the system applies a multi-tier set of models to the relevant text extracted during the input stage. The multi-tier set of models determines a medical procedure code and/or a medical classification code for the data. The multi-tier set of models can be machine learning models in certain embodiments. For a two-tier set of models for example, for each code type (e.g., medical classification and medical procedure), the system applies a first-level model. The first-level models determine a code category. A code category may be a group of codes, and a code category may be associated with an anatomical region (e.g., hands and feet, a gastrointestinal region, etc.) and/or procedure type in some examples. Based on the code category, the system selects a second-level model. For example, the system selects a second-level medical procedure model based on the medical procedure code category output by the first-level medical procedure model, and the system selects a second-level medical classification model based on the medical classification code category output by the first-level medical classification model. The selected second-level models then determine medical procedure codes and medical classification codes based on the data. In other example implementations including other multi-tier models, the second-level models may instead narrow down the possible codes, for example by narrowing down the codes based on the first four characters of the medical codes. Third-level models may narrow down candidate medical codes based on the first five characters, and so on until the multi-tier set of models determines the appropriate medical code. Thus, the multi-tier set of models may have any number of tiers in various example implementations.

Conceptually, this multi-tier model structure is based on the recognition that a machine learning model can be more accurate if it is specialized and trained on specific anatomical regions and/or procedure types, rather than being a general-purpose model for all regions of the body. As an example, if a medical document such as physician's note describes a medical procedure and diagnosis relating to a broken finger, a specially trained model can more accurately predict medical procedure codes and medical classification codes when is it trained on terminology and codes relating to the hands and feet. Therefore, the first-level model evaluates the medical document to first determine that it relates to the category of hands and feet, and then the second-level model is selected to once again process the physician's note in order to determine the appropriate medical procedure codes or medical classification codes.

Similarly, the structure shown above also divides the processing by code type (e.g., medical procedure codes vs. medical classification codes) because a model trained to generate only one type of code will be more accurate than a model trained to generate both medical procedure codes and medical classification codes. In some embodiments, the models applied during the processing stage are large language models. For example, the models may use a pre-trained Bidirectional Encoder Representations from Transformers (BERT)-based model. Furthermore, the models may be fine-tuned using supervised learning for the task that they are to perform. For example, the first-level medical procedure model may be fine-tuned to classify medical procedure code (e.g., CPT code) categories. In some embodiments, for a given input data, the system may determine a plurality of different medical procedure codes and medical classification codes.

During the output stage, the system outputs the medical procedure code(s) and medical classification code(s) determined from the data. For example, the system can output one or more pairs that include a CPT code and an ICD-10 code. One CPT code may be paired with multiple ICD-10 codes in certain embodiments. Thus, the same CPT code may be in multiple pairs of PCT and ICD-10 codes. The system may also determine a confidence score associated with medical codes and/or pairs of medical codes. The confidence score indicates the confidence the respective selected code is correct and is based on a likelihood output by the models in the processing stage. The likelihood output by the models may be based at least in part on the historical accuracy given the associated code pattern the model used to determine the respective medical code. The confidence score may be altered by a set of use case-specific rules in some embodiments. The multi-tier model can be improved by training and otherwise fine-tuning the model based on the outputs the model generates.

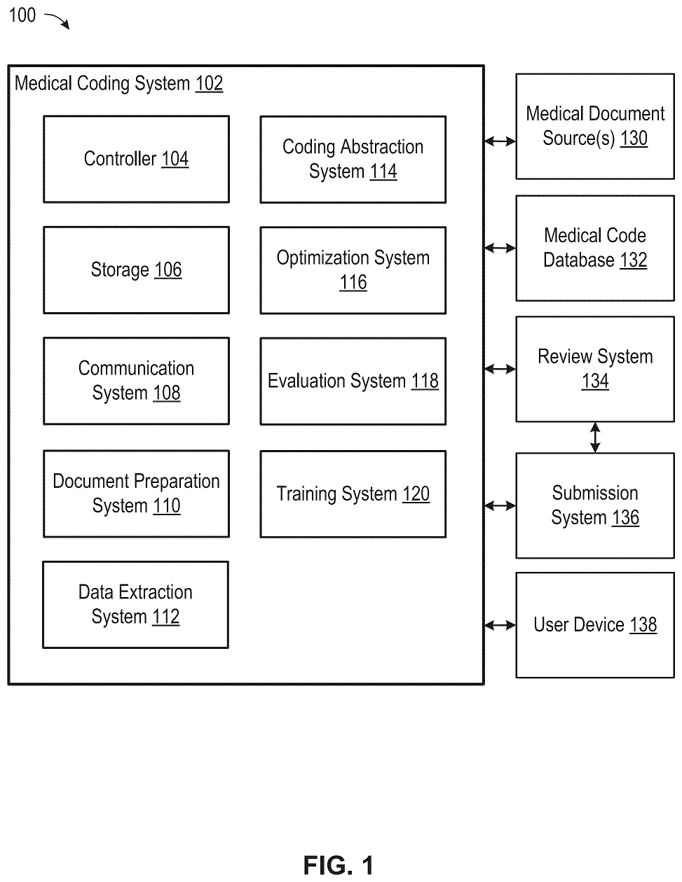

FIG. 1 is a block diagram of an operating environment 100 for automatically determining medical codes. The operating environment 100 includes a medical coding system 102 with a controller 104, a storage 106, a communication system 108, a document preparation system 110, a data extraction system 112, a coding abstraction system 114, an optimization system 116, an evaluation system 118, and a training system 120. The operating environment 100 can also include medical document source(s) 130, a medical code database 132, a review system 134, a submission system 136, and a user device 138.

The controller 104 can control the operation of the medical coding system 102 and the medical coding system 102 components. The storage 106 can store instructions for the operation of the medical coding system 102, medical code information, medical document information, medical coding models, medical data, and/or the like. The controller 104 may therefore execute instructions the storage 106 stores to control the operation of the medical coding system 102. In some embodiments, the medical coding system 102 can access external or remote storages, such as remote servers.

The communication system 108 enables the medical coding system 102 to communicate with local devices and remote devices, such as via a network. The communication system 108 can include Wi-Fi capabilities, cellular capabilities, and/or the like. Thus, the medical coding system 102 may communicate with the medical document source(s) 130, a medical code database 132, the review system 134, the submission system 136, the user device 138, and/or the like via the communication system 108.

The medical document source(s) 138 may be systems or other sources that send medical documents to the medical coding system 102, such as a medical entity's device, a medical professional's device, and/or the like. The medical code database 132 may store medical codes, such as CPT and ICD-10 codes, for the medical coding system 102 to use. For example, the medical coding system 102 may ensure any codes assigned are accurate and up to date via the medical code database 132. The review system 134 may be a system for review of the medical coding the medical coding system 102 generates. For example, a manual reviewer may use the review system 134 to evaluate the medical coding the medical coding system 102 generates. The submission system 136 may be a system for receiving finalized medical coding for processing, such as billing. The user device 138 may be a device that requests access to medical coding information or sends commands to the medical coding system 102. For example, the user device 138 may request medical documents to be evaluated and assigned medical codes, to review generated medical codes, and/or the like. In some examples, the user device 138 is associated with a healthcare entity or medical professional.

The document preparation system 110 can prepare medical documents for automatic medical coding. The document preparation system 110 may prepare medical documents as the medical coding system 102 receives the documents from the medical document source(s), in response to a request to prepare specified medical documents, and/or the like. The document preparation system 110 can perform one or more processes for making data in medical documents usable during the automatic medical coding process, including performing optical character recognition (OCR), natural language processing (NLP), and/or other processing tasks. The medical documents can include free-form text the document preparation system 110 can prepare using OCR or extracted from the text layer using another process, can include Hypertext Markup Language (HTML) the document preparation system 110 can prepare, and/or the like.

The document preparation system 110 may prepare the medical document for the data extraction system 112, and the data extraction system 112 may identify relevant data in the prepared medical documents. The data extraction system 112 can identify relevant sections of data in medical documents and extract the data from the relevant sections. Thus, the data extraction system 112 may identify relevant data from different formats of medical documents since medical document formats may vary by provider, medical professional, and/or the like. The data extraction system 112 may also identify relevant metadata associated with the medical documents, such as the date of service, the medical provider, the referring provider, patient information, and/or the like. In some embodiments, the document preparation system 110 and the data extraction system 112 may operate simultaneously or otherwise as a single system.

Once the data extraction system 112 extracts the relevant data from the prepared medical documents, the coding abstraction system 114 can identify and assign medical codes based on the extracted data. The coding abstraction system 114 can utilize a multi-tier set of models with the relevant data the data extraction system 112 extracted used as input. The multi-tier set of models determines a medical procedure code and/or a medical classification code for one or more portions of the relevant data. The multi-tier set of models can be machine learning models in certain embodiments.

For each code type (e.g., medical classification codes and medical procedure codes), the coding abstraction system 114 applies a first-level model, and the first-level models determine a code category. There may be any number of first-level models, for example based on the number of medical code types to be determined. A code category may be a group of codes, and a code category may be associated with an anatomical region (e.g., hands and feet, a gastrointestinal region, etc.) and/or procedure type in some examples. Based on the code category, the system selects a second-level model. For example, the system selects a second-level medical procedure model based on the medical procedure code category output by the first-level medical procedure model, and the system selects a second-level medical classification model based on the medical classification code category output by the first-level medical classification model. The selected second-level models then determine medical procedure codes and medical classification codes based on the data. Because medical procedure codes identify services rendered and medical classification codes represent patient diagnoses, the coding abstraction system 114 may pair the associated medical procedure codes and medical classification codes based on the relationships between the medical procedure codes and medical classification codes.

The coding abstraction system 114 can also determine and assign confidence scores to the determined medical procedure and medical classification codes to indicate the confidence level that the coding abstraction system 114 selected the correct codes. If the confidence level of one or more codes is below a threshold, the medical coding system 102 may send the codes to a review system 134 for evaluation.

In certain embodiments, the medical coding system 102, including the coding abstraction system 114, can utilize artificial intelligence and/or machine learning to automatically prepare medical documents, extract data from prepared medical documents, determine medical codes based on medical documents, and so on as described herein. Machine learning techniques are generally used for receiving data as input and recognizing complex patterns in the data. In various implementations, the medical coding system 102 may utilize supervised, unsupervised, and/or semi-supervised machine learning models. Supervised learning involves the use of a training set of data used to train the model to apply labels to the input data. For example, the training data may include sample telemetry that has already been labeled as being indicative of an acceptable performance or unacceptable performance. Unsupervised techniques may not utilize a training set of labels. While a supervised learning model may look for previously seen patterns that have been labeled as such, an unsupervised model may instead look to whether there are sudden changes or patterns in the behavior of the metrics. Semi-supervised learning models are a mixed approach that use a reduced set of labeled training data.

Example machine learning techniques that the medical coding system 102 can employ may include Nearest Neighbor (NN) techniques (e.g., k-NN models, replicator NN models, etc.), statistical techniques (e.g., Bayesian networks, etc.), clustering techniques (e.g., k-means, mean-shift, etc.), neural networks (e.g., reservoir networks, artificial neural networks, etc.), Support Vector Machines (SVMs), Generative Adversarial Networks (GANs), Long Short-Term Memory (LSTM), logistic or other regression, Markov models or chains, Principal Component Analysis (PCA) (e.g., for linear models), Singular Value Decomposition (SVD), Multi-Layer Perceptron (MLP) Artificial Neural Networks (ANNs) (e.g., for non-linear models), replicating reservoir networks (e.g., for non-linear models, typically for timeseries), random forest classification, and/or the like.

Once the coding abstraction system 114 determines medical codes and confidence scores, the optimization system 116 may evaluate the generated medical codes to optimize or otherwise improve the output of the medical coding system 102. For example, the optimization system 116 can add, remove, and otherwise edit the generated medical codes to prevent duplicate codes, overlapping codes, and/or the like. In further examples, the optimization system 116 may apply Correct Code Initiative (CCI) edits to prevent overpayment of duplicative or overlapping medical codes. The optimization system 116 can additionally add medical procedure modifiers (e.g., CPT modifiers) used to supplement the information of the medical procedure codes, adjust care descriptions to provide additional details concerning the procedure or service provided, and the like. The optimization system 116 may use medical classification codes (e.g., ICD-10 codes) associated with a given medical procedure code to add medical procedure modifiers in some example implementations.

The medical coding system 102 may finalize the generated medical code group after optimizing the medical codes. In some embodiments, the finalized medical coding may comprise a medical coding report, as will be described in further detail herein. The medical coding system 102 may send the finalized medical codes to one or more systems for use, such as for billing. The medical coding system 102 may determine whether to send the finalized codes to the review system 134 for evaluation or to send the codes directly to other devices based on the confidence scores. Thus, the medical coding system 102 can send the codes to the submission system 136, the review system 134, the user device 138, and/or the like.

The medical coding system 102 can receive feedback regarding the accuracy of the generated codes, the accuracy of identifying relevant data, the completeness of the medical coding, and/or the like. For example, the medical coding system 102 can receive Remittance Advice (RA) from payors, feedback from the review system 134, feedback from the submission system 136, feedback from the user device 138, and/or the like. The evaluation system 118 can use the feedback to improve the operation of the medical coding system 102 for subsequent determinations of medical codes, including enabling the coding abstraction system 114 to more accurately determine medical codes and confidence scores.

The training system 120 may train or otherwise fine-tune the multi-tier model and/or other machine learning models the medical coding system 102 uses to improve the models using additional data. The re-training can be periodic and incorporate data processed by the medical coding system 102 previously, periodic internal and collaborative auditing of customer results, feedback regarding the accuracy of the generated medical codes of the medical coding system 102, and/or the like.

The medical coding system 102 may include more or fewer systems in other examples. For example, the medical coding system 102 may include a display that displays interfaces, such as Graphical User Interface (GUI) views. The interfaces may include user login elements, medical document upload elements, medical coding review elements, user authentication elements, and/or the like. Additionally, one or more components of the medical coding system 102 may be combined and/or perform other functions in other embodiments. Some processes may also be done by another system in some example implementations, such as the medical coding system 102 receiving preprocessed medical documents.

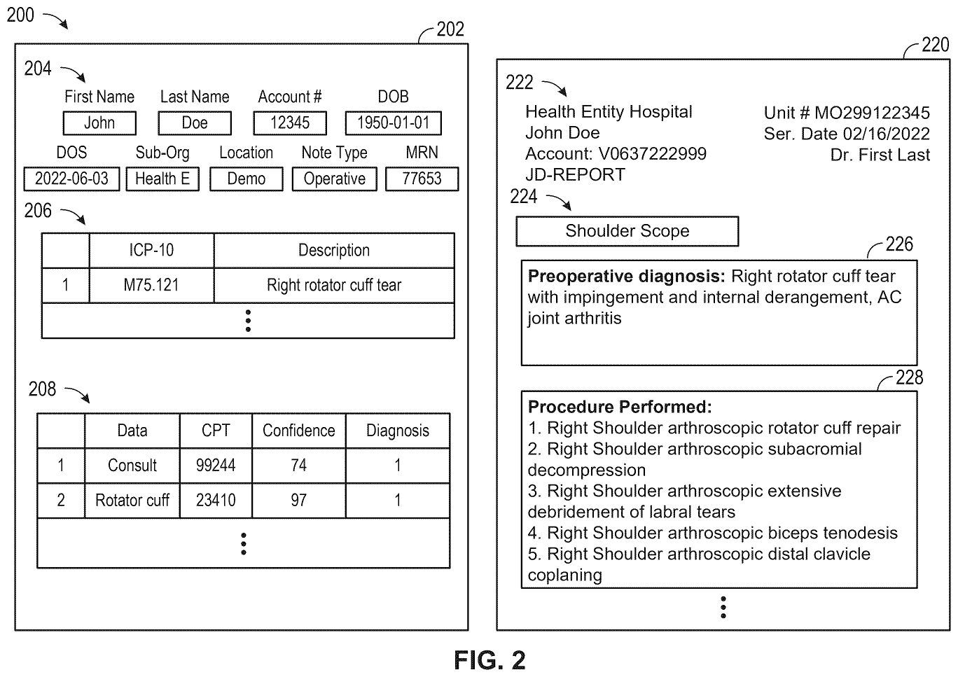

FIG. 2 is a diagram of finalized medical coding 200 including a medical coding report 202 generated based on a medical document 220. The medical coding report 202 includes metadata 204, a medical classification code section 206, and a medical procedure code section 208. The medical document 220 includes a header section 222, a title section 224, a preoperative diagnosis section 226, and a procedure performed section 228.

The medical coding system 102 may prepare the medical document 220 and extract relevant data from the medical document 220. For example, the data extraction system 112 may identify relevant metadata in the header section 222, identify data relevant to the procedure and diagnosis type in the title section 224, and identify relevant data in the preoperative diagnosis section 226, and the procedure performed section 228. In some examples, there may be sections with no relevant data, and the data extraction system 112 may not extract any data from irrelevant sections.

Once the data extraction system 112 identifies the relevant data and sections, the coding abstraction system 114 can use the relevant data (e.g., data extracted from the header section 222, title section 224, the preoperative diagnosis section 226, and the procedure performed section 228) to identify the metadata 204, medical procedure codes, medical classification codes, and the like, and include the information and medical codes in the medical coding report 202. For example, the coding abstraction system 114 can use data from the title section 224, the preoperative diagnosis section 226, and the procedure performed section 228 to identify the associated anatomical region and/or procedure type for use by the first level model to identify the code category and select second-level models for the medical classification codes and medical procedure codes. The coding abstraction system 114 can use data from the preoperative diagnosis section 226 and any relevant data in the other sections to determine medical classification codes and can use the data from the procedure performed section 228 and other relevant sections to determine medical procedure codes.

The coding abstraction system 114 can then generate the medical coding report 202 based on the evaluation of the data in the medical document 220. The metadata 204 can include information associated with the medical documents, such as the date of service, the medical provider, the referring provider, patient information, and/or the like. The medical classification code section 206 can include determined medical classification codes, a description of the data used to determine the code, a confidence level, and/or the like. The medical procedure code section 208 can include determined medical procedure codes, a description of the data used to determine the code, a confidence level, the medical classification code pair, and/or the like. Each medical coding report 202 can include any amount of metadata 204, any amount of medical classification codes in the medical classification code section 206, and any amount of medical procedure codes in the medical procedure code section 208 depending on the data available in the medical document 220. In some embodiments, multiple medical documents 220 may be used to generate a single medical coding report 202, such as multiple medical documents 220 related to a single patient. While the medical coding report 202 is shown as a viewable document in this example, the medical coding report 202 may be in a different format, file type, etc. in other examples. For example, the medical coding report 202 may be a sequence of bits indicating the information for other devices in some implementations.

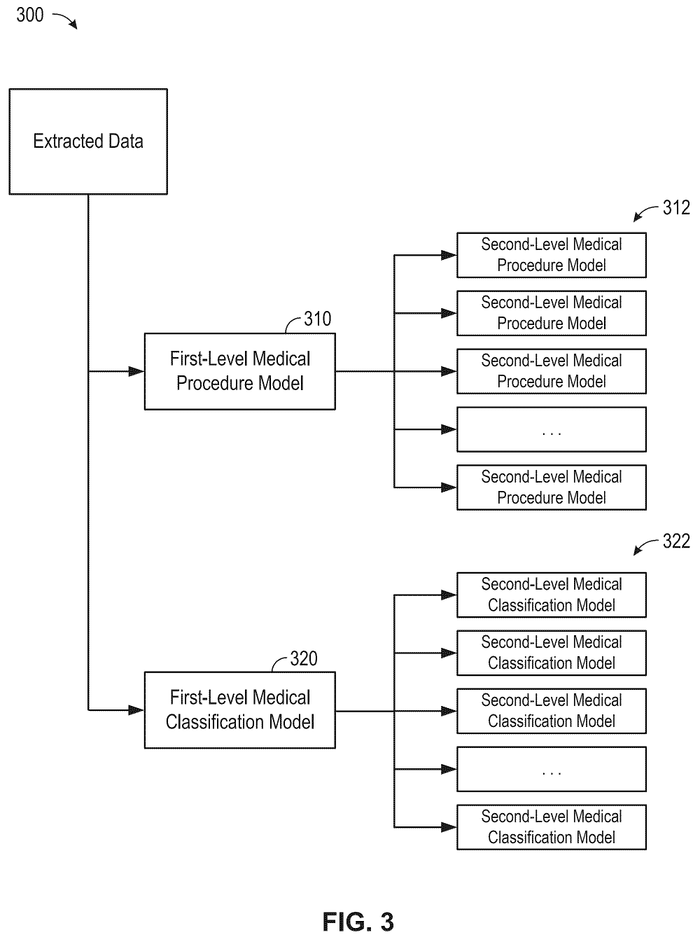

FIG. 3 is a block diagram of a multi-tier set of models 300 for determining medical codes. The multi-tier set of models 300 can include a first-level medical procedure model 310, second-level medical procedure models 312, a first-level medical classification model 320, and second-level medical classification models 322. Thus, in this illustrated example, the multi-tier set of models 300 is a two-tier set of models. However, the multi-tier set of models 300 may include more tiers in other embodiments. The coding abstraction system 114 can use the multi-tier set of models 300 to determine medical procedure codes and medical classification codes based on medical documents, such as the medical document 220.

The first-level medical procedure model 310 and the first-level medical classification model 320 can receive extracted data from one or more medical documents (e.g., as extracted by the data extraction system 112) and evaluate the extracted data to determine a code category. A code category may be a group of codes, and a code category may be associated with an anatomical region and/or procedure type in some examples. For example, various models the second-level medical procedure models 312 and the second-level medical classification models 322 may be related to hands, spine, feet, shoulders, skin, head, abdomen, heart, legs, arms, neck, orthopedics, anesthesiology, cardiology, dermatology, bariatrics, allergy and immunology, gastroenterology, hematology, infectious disease, neurology, urology, toxicology, and/or the like.

Based on the code category, the first-level medical procedure model 310 selects one of the second-level medical procedure models 312 and the first-level medical classification model 320 selects one of the second-level medical classification models 322. The selected second-level medical procedure model 312 can then use the extracted data to determine one or more medical procedure codes, and the selected second-level medical classification model 322 can use the extracted data to determine one or more medical classification codes. The medical coding system 102 can also determine a confidence score for the medical procedure codes and medical classification codes. In other example implementations utilizing other multi-tier models, the second-level models may instead narrow down the possible codes, for example by narrowing down the codes based on the first four characters of the medical codes. Third-level models may further narrow down possible medical codes, for example based on the first five characters of the possible medical codes, and so on until the multi-tier set of models determines the appropriate medical code. Thus, the multi-tier set of models 300 may have any number of tiers in various example implementations.

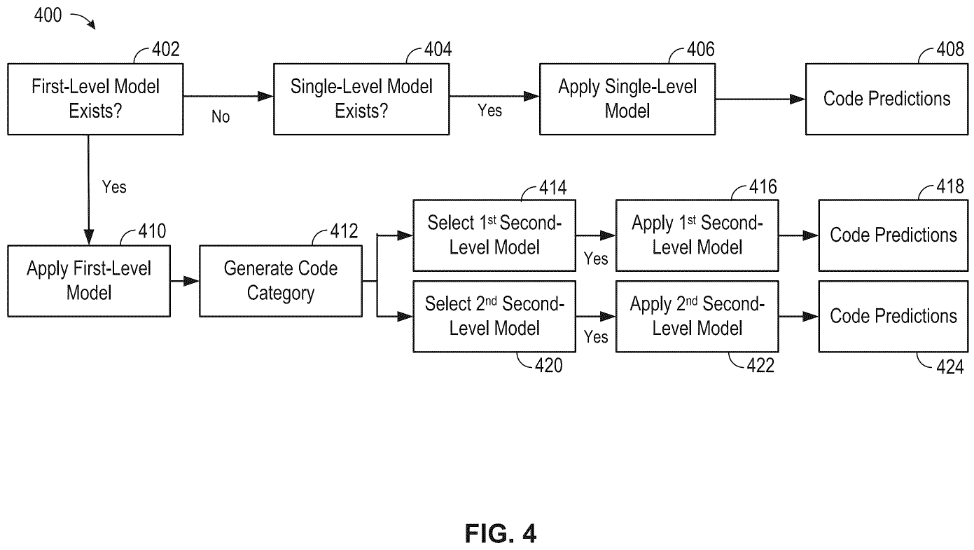

FIG. 4 is a flow chart of a method 400 for selecting a model for determining medical codes. The medical coding system 102 and/or other devices can use the method 400 to determine a model to use for determining medical codes. The method 400 can begin at operation 402, and it can be determined whether a first-level model of a multi-tier set of models exists. For example, the medical coding system 102 determines whether the first-level medical procedure model 310 and/or the first-level medical classification model 320 is deployed or otherwise available to be used by the medical coding system 102. If there is no first-level model available, the method 400 can proceed to operation 404. In operation 404, it is determined whether a single-level model exists. For example, the medical coding system 102 determines whether there is a single-level model the medical coding system 102 can use for determining medical codes. If the medical coding system 102 identifies a single-level model, the method 400 can proceed to operation 406, and the medical coding system 102 can input relevant data into the single-level model. In operation 408, the single-level model may output determined medical codes.

If the medical coding system 102 identifies a first-level model in operation 402, the method 400 can proceed to operation 410. In operation 410, the medical coding system 102 can input relevant data into the first-level model (e.g., first-level medical procedure model 310 and/or the first-level medical classification model 320). In operation 412, the first-level model can generate a code category. In operation 414, a first second-level model is identified based on the code category. For example, the first second-level model may be a model for determining medical procedure codes. In operation 416, the relevant data is input into the first second-level model. In operation 418, the first second-level model determines medical codes. In addition to operation 414, operation 420 may occur after operation 412. In operation 420, a second second-level model is identified based on the code category. For example, the first second-level model may be a model for determining medical classification codes. Thus, both medical procedure codes and medical classification codes may be determined in the method 400. In operation 422, the relevant data is input into the second second-level model. In operation 424, the second second-level model determines medical codes. Various amounts of second-level models may be identified and used in other examples.

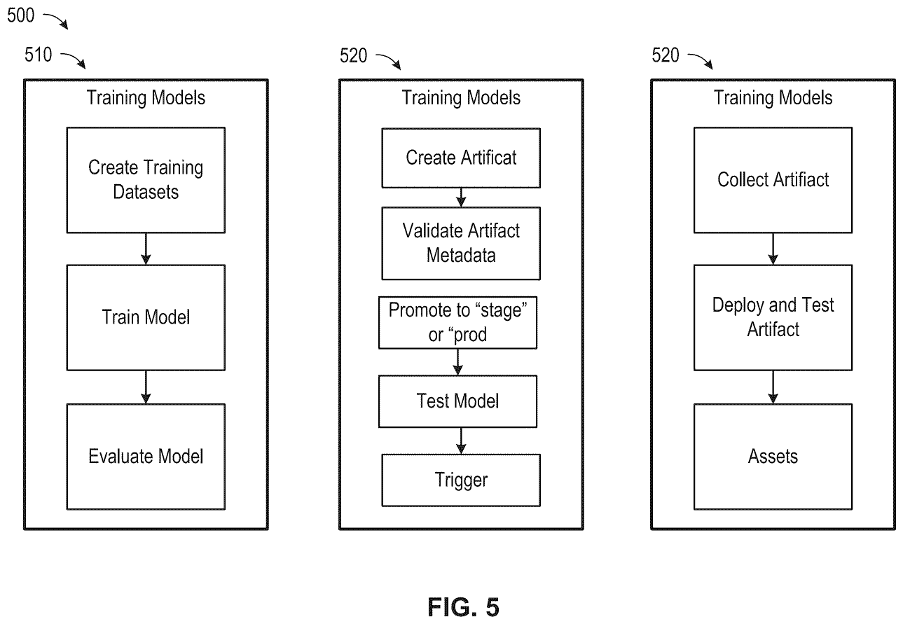

FIG. 5 is a diagram of a deployment 500 of models for determining medical codes. The deployment 500 can include training models 510, a model registry 520, and an orchestrator 530. The deployment 500 can include virtual models that represent a model artifact and that can have multiple versions, but only one version is deployed at a time. The deployment 500 can also include physical models that are actual versions of a model artifacts or virtual models. Physical models can include corresponding metadata on how the model was generated and the path where the archive can be found for deployment. The training models 510 can include candidate models that are undergoing training. Training can include creating training values and datasets, training the candidate models using the training values and datasets, and evaluating the trained models to determine if the candidate model can be deployed. Physical Models are the end product of the model training process.

The model registry 520 can include a list of virtual models, including any multiple versions of the virtual models. Each version may be associated to a different physical model. For each Virtual Model, there will be one artifact version/Physical Model with the aliases “prod” and “stage” identifying which Physical Model is actually deployed in each environment. The orchestrator 530 can manage the input of medical documents for preparations, data extraction, determining medical codes, and/or other operations.

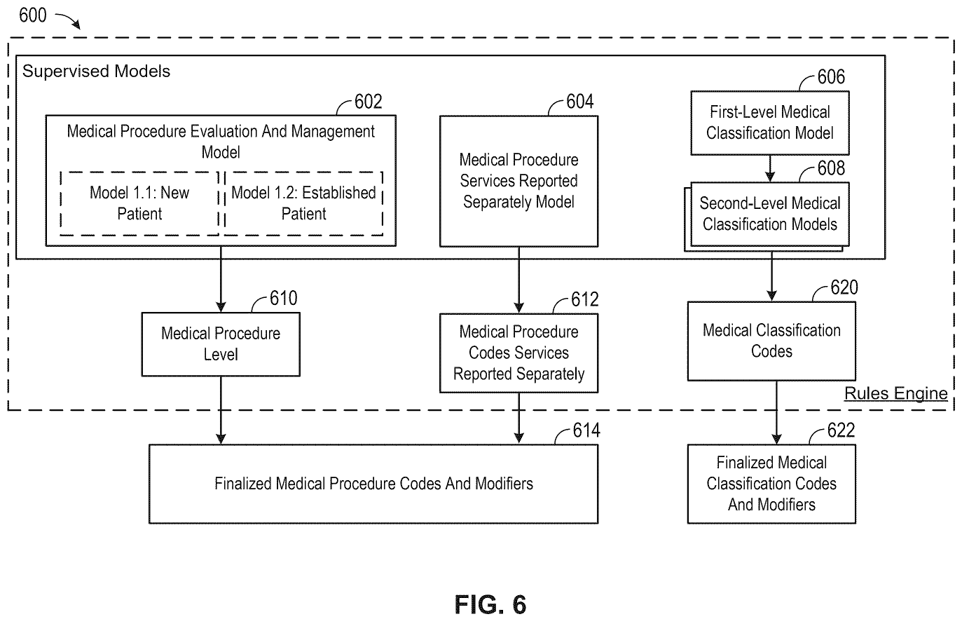

FIG. 6 is a block diagram of a model set 600 for determining evaluation and management medical codes in accordance with aspects of the present disclosure. Medical documents may be associated with three primary use cases in some embodiments: operative documents describing procedures such as surgeries, evaluation and management documents describing office visits or other outpatient services, and diagnostic documents associated with diagnosing injuries and illnesses. For operative documents, the coding abstraction system 114 may identify code categories associated with the procedure and/or anatomical regions associated with the procedure. For the evaluation and management documents, the coding abstraction system 114 may identify a code category for medical procedure evaluation and management codes and a code category for medical procedure services reported separately. There may be a single or otherwise general second-level model for evaluation and management and services reported separately.

The model set 600 can include a medical procedure evaluation and management model 602, a medical procedure services reported separately model 604, a first-level medical classification model 606, and second-level medical classification models 608. The medical procedure evaluation and management model 602 can be Medical Decision Making (MDM) and/or time-based for determining a medical procedure level 610 (e.g., CPT level). For example, if an MDM value is higher than the time-based value, the medical procedure level 610 may be based on three MDM elements: problems, data, and risk. To determine the medical procedure level 610, there may be different categories with criteria that must be met. For example, to determine the medical procedure level 610 is moderate, a first category may include review of prior external documents from unique sources, review of the results of each unique test, ordering of each unique test, and assessment requiring an independent historian. A second category may require independent interpretation of a test performed by another medical profession. A third category may require discussion of management or test interpretation with an external medical professional.

The medical procedure services reported separately model 604 may identify medical procedure codes of services reported separately 612 from the evaluation and management data. The medical procedure evaluation and management model 602 and the medical procedure services reported separately model 604 can generate final medical procedure codes and modifiers 614 based on the medical procedure level 610 and the medical procedure codes of services reported separately 612. The first-level medical classification model 606 and second-level medical classification models 608 can generate full medical classification codes 620 and final medical classification codes and modifiers 622 based on the full medical classification codes.

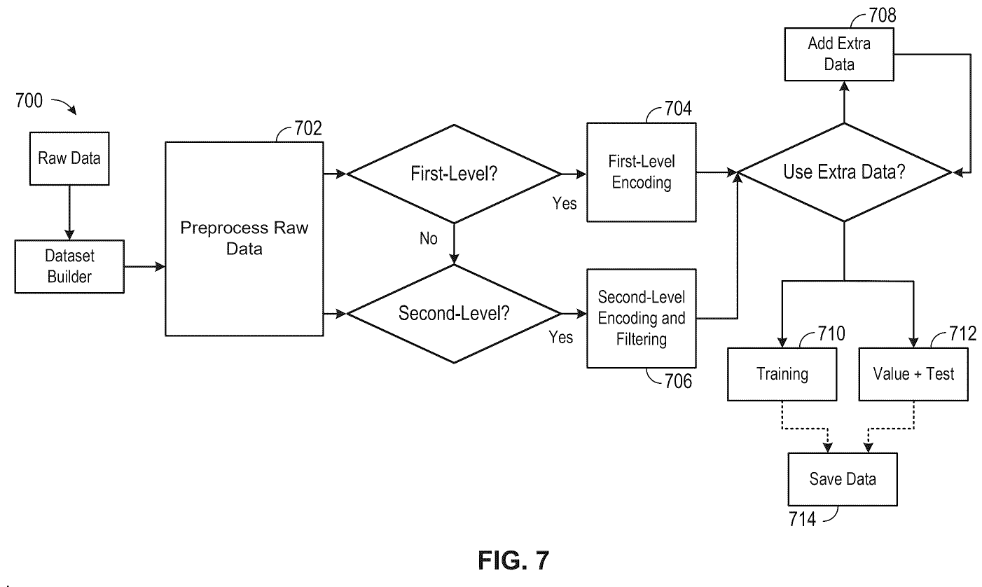

FIG. 7 is a flow chart of a method 700 for training models for determining medical codes in accordance with aspects of the present disclosure. The method 700 can start with processing raw training data in operation 702. Preprocessing the raw data can include cleaning the raw training data, such as by removing small medical documents (e.g., less than fifty characters), applying filters, creating code categories and associating the categories with second-level models (e.g., identifying the correct second-level model or creating a new second-level model), transforming a code list to columns with one document-code pair per row, and/or the like. For evaluation and management training data, non-evaluation and management codes can be filtered out, document sections can be filtered, and data can be regrouped with the remaining sections.

If a first-level model is being trained, the method 700 can proceed to operation 704. In operation 704, encoding, such as one-hot encoding, can be performed, including labelling code categories or otherwise associating them with second-level models, spreading the labels through columns with one document per row, assigning binary values for present codes (e.g., 1 if the code is present, 0 if the code is not present) and/or the like. If a second-level model is being trained, the method 700 can proceed to operation 706. In operation 706, the data can be filtered so only codes associated second-level model will be determined, and a code minimum frequency is set. Encoding, such as one-hot encoding, can also be performed, including setting one document per row, one column per code, setting binary values to indicate the codes that are present, and/or the like.

After operation 704 or operation 706, it is determined whether extra data is used. If extra data is used, the method 700 proceeds to operation 708. In operation 708, extra data can be added for training. If extra data is not used, the method 700 proceeds to operation 710 and operation 712. In operation 710, the model can be trained using the processed data (e.g., from operation 704 or operation 706). In operation 712, the model can output determined codes for comparison to expected values. Thus, the performance of the model can be evaluated to determine if the model can be deployed for use determining medical codes. In operation 714, the dataset and/or metadata can be saved, for example for future testing.

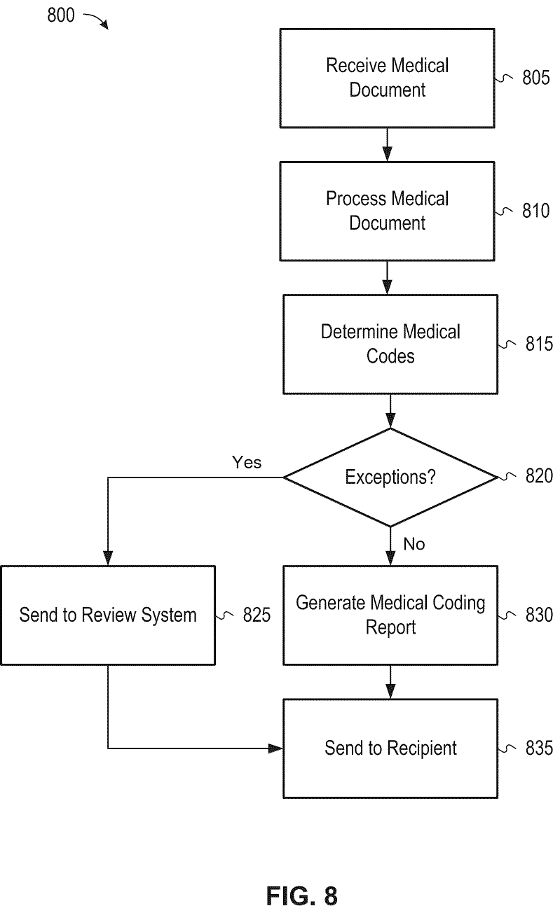

FIG. 8 is a flow chart of a method 800 for automatically determining medical codes based on medical documents. The method 800 may begin at operation 805, and one or more medical documents are received. For example, the medical coding system 102 receives one or more medical documents, such as from the medical document source(s) 130. In some embodiments, the medical coding system 102 may request one or more documents from another system or access stored documents, whether stored locally or remotely.

In operation 810, the medical documents are processed. For example, the medical coding system 102 prepares the medical documents, identifies relevant data, and extracts the relevant data. The data extraction system 112 may also identify relevant metadata associated with the medical documents, such as the date of service, the medical provider, the referring provider, patient information, and/or the like. In some embodiments, the document preparation system 110 and the data extraction system 112 may operate simultaneously or otherwise as a single system to process medical documents. The medical coding system 102 may user OCR, NLP, and/or the like to process the one or more medical documents in some embodiments.

In operation 815, medical codes are determined. For example the medical coding system 102 determines one or more medical codes using a multi-tier plurality of models with the extracted data as input. In some embodiments, the medical coding system 102 can determine medical codes by applying a first-level medical procedure model to the data to determine a medical code category. Based on the medical code category, the medical coding system 102 can select a second-level medical model from a plurality of second-level medical models and apply the selected second-level medical model to the data to determine the one or more medical codes. In certain embodiments, determining the one or more medical codes can comprise applying a first-level medical procedure model to the data to determine a medical code category. Based on the medical code category, one or more intermediate-level medical models from a plurality of intermediate-level medical models can be applied to the data to narrow down candidate medical codes. Then a final-level medical model can be applied to the data to determine the one or more medical codes. The medical code category can be associated with an anatomical region, a medical procedure, and/or the like.

In certain embodiments, the medical coding system 102 can determine medical codes by applying a first-level medical procedure model to data to determine a medical procedure code category. Based on the medical procedure code category, the medical coding system 102 selects a second-level medical procedure model from a plurality of second-level medical procedure models and applies the selected second-level medical procedure model to the data to determine a medical procedure code. The medical coding system 102 can also apply a first-level medical classification model to the data to determine a medical classification code category. Based on the medical classification code category, the medical coding system 102 selects a second-level medical classification model from a plurality of second-level medical classification models and applies the selected second-level medical classification model to the data to determine a medical classification code. In some embodiments, the medical procedure code is a CPT code, and the medical classification code is an ICD-10 code. The medical procedure code category and the medical classification code category can be associated with an anatomical region, a medical procedure, and/or the like. In certain embodiments, the medical coding system 102 can create a pair of the medical procedure code and the medical classification code, such as when the two codes are related. Additionally, the multi-tier plurality of models can be pre-trained models (e.g., BERT models) that are fine-tuned using supervised learning to classify one or more of medical procedure codes, medical classification codes, and/or the like. In some embodiments, the medical coding system 102 also assigns confidence scores to the determined medical codes. For example, the medical coding system 102 may assign a confidence score to each medical code based on the determined likelihood the multi-tier plurality of models determined the correct code for the respective data.

In decision 820, it is determined whether there are exceptions in the determined medical codes. For example, the medical coding system 102 determines whether any confidence scores are below a threshold confidence level to trigger a review. If the medical coding system 102 determines there is one or more exceptions, the method 800 proceeds to operation 825. In operation 825, the medical coding system 102 sends the determined medical codes to a review system, such as the review system 134. The medical coding system 102 may send a medical coding report, such as the medical coding report 202, in some embodiments. The review system 134 can evaluate the medical codes, such as the medical codes with confidence scores below the threshold, before finalizing a medical coding report. If the medical coding system 102 determines there are no exceptions, the method 800 proceeds to operation 830. In operation 830, the medical coding system 102 generates a medical coding report, such as the medical coding report 202. In operation 835, the medical coding report is sent to a recipient, such as the submission system 136.

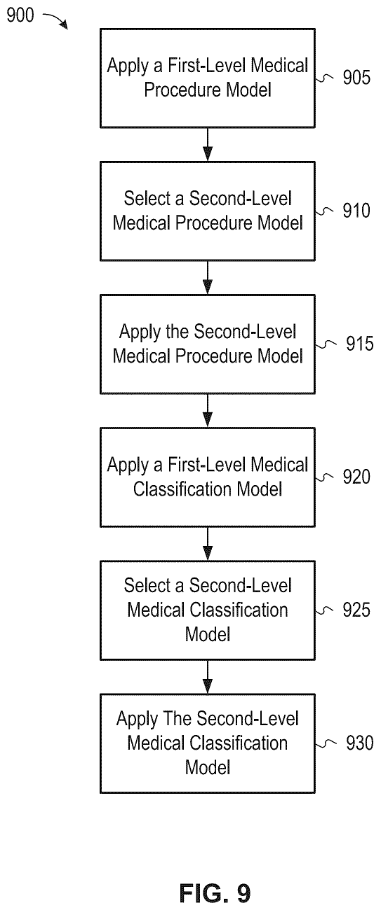

FIG. 9 is a flow chart of a method 900 for automatically determining medical codes. The method 900 may be a part of the method 800 in some embodiments. For example, operation 815 may comprise the operations of the method 900. Method 900 may begin at operation 905. In operation 905, a first-level medical procedure model is applied. For example, the medical coding system 102 applies the first-level medical procedure model 310 to data to determine a medical procedure code category. The medical coding system 102 may extract the data from a medical document, for example by performing OCR, NLP, identifying relevant sections of the medical document, and/or the like. The medical procedure code category may be associated with an anatomical region, a medical procedure, and/or the like.

In operation 910, a second-level medical procedure model is selected. For example, the medical coding system 102 selects one of the second-level medical procedure models 312 based on the determined medical procedure code category. In operation 915, the second-level medical procedure model is applied. For example, the medical coding system 102 applies the selected second-level medical procedure model 312 to the data to determine a medical procedure code. In some embodiments, the medical procedure code is a CPT code.

In operation 920, a first-level medical classification model is applied. For example, the medical coding system 102 applies the first-level medical classification model 320 to data to determine a medical classification code category. The medical classification code category may be associated with an anatomical region, a medical procedure, and/or the like.

In operation 925, a second-level medical classification model is selected. For example, the medical coding system 102 selects one of the second-level medical classification models 322 based on the determined medical classification code category. In operation 930, the second-level medical classification model is applied. For example, the medical coding system 102 applies the selected second-level medical classification model 322 to the data to determine a medical classification code. In some embodiments, the medical classification code is an ICD-10 code. The multi-tier plurality of models can be pre-trained models that are fine-tuned using supervised learning to classify medical procedure codes, medical classification codes, and/or the like.

In some embodiments, a confidence score can be assigned to the medical procedure code, the medical classification code, or both. The confidence score(s) can be output, such as included in a medical coding report or otherwise sent to another system. In certain embodiments, a pair may be created with the medical procedure code and the medical classification code.

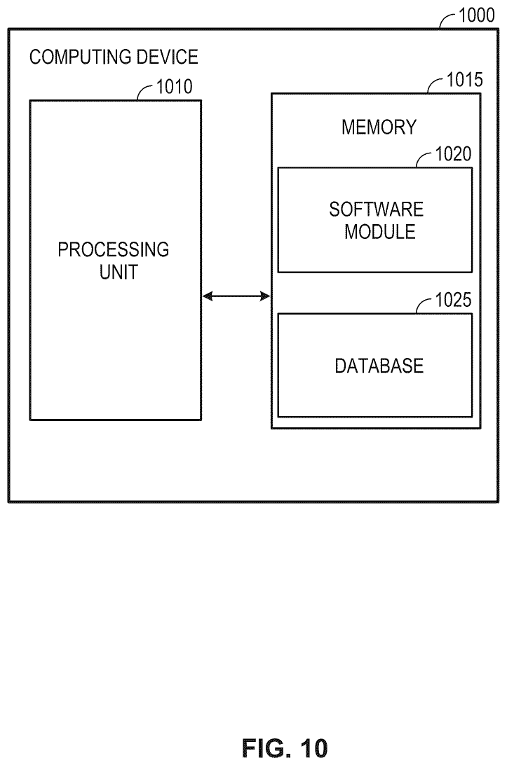

FIG. 10 is a block diagram of a computing device 1000. As shown in FIG. 10, computing device 1000 may include a processing unit 1010 and a memory unit 1015. The memory unit 1015 may include a software module 1020 and a database 1025. While executing on the processing unit 1010, software module 1020 may perform, for example, processes for automatically determining medical codes with respect to FIG. 1, FIG. 2, FIG. 3, FIG. 4, FIG. 5, FIG. 6, FIG. 7, FIG. 8, and FIG. 9. Computing device 1000, for example, may provide an operating environment for the medical coding system 102, the controller 104, the storage 106, the communication system 108, the document preparation system 110, the data extraction system 112 the coding abstraction system 114, the optimization system 116, the evaluation system 118, the training system 120, the medical document source(s) 130, the medical code database 132, the review system 134, the submission system 136, the user device 138, and the like. The medical coding system 102, the controller 104, the storage 106, the communication system 108, the document preparation system 110, the data extraction system 112 the coding abstraction system 114, the optimization system 116, the evaluation system 118, the training system 120, the medical document source(s) 130, the medical code database 132, the review system 134, the submission system 136, the user device 138, and the like may operate in other environments and are not limited to computing device 1000.

Computing device 1000 may be implemented using a Wi-Fi access point, a tablet device, a mobile device, a smart phone, a telephone, a personal computer, a network computer, a mainframe, a router, a switch, a server cluster, a network storage device, a network relay device, or other similar microcomputer-based device. Computing device 1000 may comprise any computer operating environment, such as hand-held devices, multiprocessor systems, microprocessor-based or programmable sender electronic devices, minicomputers, mainframe computers, and the like. Computing device 1000 may also be practiced in distributed computing environments where tasks are performed by remote processing devices. The aforementioned systems and devices are examples, and computing device 1000 may comprise other systems or devices.

Referring to the above process generally, it is noted that certain aspects may be performed in different orders. Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed invention.

The example embodiments described herein may be implemented using hardware, software, or a combination thereof and may be implemented in one or more computer systems or other processing systems. However, the manipulations performed by these example embodiments were often referred to in terms, such as entering, which are commonly associated with mental operations performed by a human operator. No such capability of a human operator is necessary in any of the operations described herein. Rather, the operations may be completely implemented with machine operations. Useful machines for performing the operation of the example embodiments presented herein include general purpose digital computers or similar devices.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium.

More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to, mechanical, optical, fluidic, and quantum technologies. In addition, embodiments of the disclosure may be practiced within a general-purpose computer or in any other circuits or systems.

Embodiments of the disclosure may be practiced via a system-on-a-chip (SOC) where each or many of the elements illustrated in FIG. 1 may be integrated onto a single integrated circuit. Such an SOC device may include one or more processing units, graphics units, communications units, system virtualization units and various application functionality all of which may be integrated (or “burned”) onto the chip substrate as a single integrated circuit. When operating via an SOC, the functionality described herein with respect to embodiments of the disclosure, may be performed via application-specific logic integrated with other components of computing device 1000 on the single integrated circuit (chip).

From a hardware standpoint, a CPU typically includes one or more components, such as one or more microprocessors, for performing the arithmetic and/or logical operations required for program execution, and storage media, such as one or more memory cards (e.g., flash memory) for program and data storage, and a random-access memory, for temporary data and program instruction storage. From a software standpoint, a CPU typically includes software resident on a storage media (e.g., a memory card), which, when executed, directs the CPU in performing transmission and reception functions. The CPU software may run on an operating system stored on the storage media, such as, for example, UNIX or Windows, iOS, Linux, and the like, and can adhere to various protocols such as the Ethernet, ATM, TCP/IP protocols and/or other connection or connectionless protocols. As is well known in the art, CPUs can run different operating systems, and can contain different types of software, each type devoted to a different function, such as handling and managing data/information from a particular source or transforming data/information from one format into another format. It should thus be clear that the embodiments described herein are not to be construed as being limited for use with any particular type of server computer, and that any other suitable type of device for facilitating the exchange and storage of information may be employed instead.

A CPU may be a single CPU, or may include plural separate CPUs, wherein each is dedicated to a separate application, such as, for example, a data application, a voice application, and a video application. Software embodiments of the example embodiments presented herein may be provided as a computer program product, or software, which may include an article of manufacture on a machine accessible or non-transitory computer-readable medium (i.e., also referred to as “machine readable medium”) having instructions. The instructions on the machine accessible or machine-readable medium may be used to program a computer system or other electronic device. The machine-readable medium may include, but is not limited to, optical disks, CD-ROMs, and magneto-optical disks or other type of media/machine readable medium suitable for storing or transmitting electronic instructions. The techniques described herein are not limited to any particular software configuration. They may find applicability in any computing or processing environment. The terms “machine accessible medium”, “machine readable medium” and “computer-readable medium” used herein shall include any non-transitory medium that is capable of storing, encoding, or transmitting a sequence of instructions for execution by the machine (e.g., a CPU or other type of processing device) and that cause the machine to perform any one of the methods described herein. Furthermore, it is common in the art to speak of software, in one form or another (e.g., program, procedure, process, application, module, unit, logic, and so on) as taking an action or causing a result. Such expressions are merely a shorthand way of stating that the execution of the software by a processing system causes the processor to perform an action to produce a result.

Additional aspects of the present disclosure are listed in the following clauses:

Clause 1. A method for determining medical codes using a multi-tier plurality of models, the method comprising: applying a first-level medical procedure model to data to determine a medical procedure code category; based on the medical procedure code category, selecting a second-level medical procedure model from a plurality of second-level medical procedure models; applying the selected second-level medical procedure model to the data to determine a medical procedure code; applying a first-level medical classification model to the data to determine a medical classification code category; based on the medical classification code category, selecting a second-level medical classification model from a plurality of second-level medical classification models; and applying the selected second-level medical classification model to the data to determine a medical classification code.

Clause 2. The method of clause 1, wherein the medical procedure code is a Current Procedural Terminology (CPT) code, and the medical classification code is an International Classification of Diseases, 10th Revision (ICD-10) code.

Clause 3. The method of clause 1, wherein the data is extracted from a medical document by performing any one of (i) optical character recognition (OCR); (ii) natural language processing (NLP); or (iii) a combination of (i) and (ii).

Clause 4. The method of clause 1, wherein the medical procedure code category and the medical classification code category are associated with any one of (i) an anatomical region; (ii) a medical procedure; or (iii) a combination of (i) and (ii).

Clause 5. The method of clause 1, further comprising: assigning a confidence score to (i) the medical procedure code; (ii) the medical classification code; or (iii) both (i) and (ii); and outputting the confidence score.

Clause 6. The method of clause 1, further comprising creating a pair with the medical procedure code and the medical classification code.

Clause 7. The method of clause 1, wherein the multi-tier plurality of models are pre-trained models that are fine-tuned using supervised learning to classify one or more of (i) medical procedure codes; (ii) medical classification codes; or (iii) both (i) and (ii).

Clause 8. A method for automatically determining medical codes, comprising: processing and extracting data from a medical document, including extracting the data from the medical document by performing any one of (i) optical character recognition (OCR); (ii) natural language processing (NLP); (iii) identifying one or more relevant sections of the medical document; or (iv) any combination of (i)-(iii); determining one or more medical codes for the medical document using a multi-tier plurality of models; assigning a confidence score to the one or more medical codes; and generating a medical coding report comprising the one or more medical codes and the confidence score.

Clause 9. The method of clause 8, wherein determining the one or more medical codes for the medical document using the multi-tier plurality of models comprises: applying a first-level medical procedure model to the data to determine a medical code category; based on the medical code category, selecting a second-level medical model from a plurality of second-level medical models; and applying the selected second-level medical model to the data to determine the one or more medical codes.

Clause 10. The method of clause 9, wherein the medical code category is associated with any one of (iv) an anatomical region; (v) a medical procedure; or (vi) a combination of (iv) and (v).

Clause 11. The method of clause 8, wherein the one or more medical codes comprises a medical procedure code and a medical classification code.

Clause 12. The method of clause 11, further comprising creating a pair with the medical procedure code and the medical classification code.

Clause 13. The method of clause 11, wherein determining the one or more medical codes for the medical document using the multi-tier plurality of models comprises: applying a first-level medical procedure model to the data to determine a medical procedure code category; based on the medical procedure code category, selecting a second-level medical procedure model from a plurality of second-level medical procedure models; applying the selected second-level medical procedure model to the data to determine the medical procedure code; applying a first-level medical classification model to the data to determine a medical classification code category; based on the medical classification code category, selecting a second-level medical classification model from a plurality of second-level medical classification models; and applying the selected second-level medical classification model to the data to determine the medical classification code.

Clause 14. The method of clause 8, wherein assigning the confidence score is based on a likelihood the multi-tier plurality of models determined the one or more medical codes correctly.

Clause 15. The method of clause 8, determining the confidence score is below a confidence threshold; and in response to determining the confidence score is below the confidence threshold, sending the medical coding report to a review system.

Clause 16. The method of clause 8, wherein determining the one or more medical codes for the medical document using the multi-tier plurality of models comprises: applying a first-level medical procedure model to the data to determine a medical code category; based on the medical code category, applying one or more intermediate-level medical models from a plurality of intermediate-level medical models to the data to narrow down candidate medical codes; and applying a final-level medical model to the data to determine the one or more medical codes.

Clause 17. A system comprising: a memory storage; and a processing unit coupled to the memory storage, wherein the processing unit is operative to: perform any of the methods of clauses 1-16.

Clause 18. A non-transitory computer-readable medium that stores a set of instructions which when executed perform a method executed by the set of instructions comprising any of the methods of clauses 1-16.

Clause 19. A system comprising: a memory storage; and a processing unit coupled to the memory storage, wherein the processing unit is operative to: process and extract data from a medical document; determine one or more medical codes for the medical document using a multi-tier plurality of models, comprising to: apply a first-level medical model to the data to determine a medical code category, and based on the medical code category, select a second-level medical model from a plurality of second-level medical models, and apply the selected second-level medical model to the data to determine the one or more medical codes; and generate a medical coding report comprising the one or more medical codes.

Clause 20. The system of clause 19, wherein to process and extract the data from the medical document comprises to: perform any one of (i) optical character recognition (OCR); (ii) natural language processing (NLP); (iii) identify one or more relevant sections of the medical document; or (iv) any combination of (i)-(iii).

Clause 21. The system of clause 19, the processing unit being further operative to: assign a confidence score to the one or more medical codes, wherein the medical coding report further comprises the confidence score.

Clause 22. The system of clause 21, the processing unit being further operative to: determine the confidence score below a confidence threshold; and in response to determining the confidence score is below the confidence threshold, send the medical coding report to a review system.

Clause 23. The system of clause 19, wherein the medical code category is associated with any one of (i) an anatomical region; (ii) a medical procedure; or (iii) a combination of (i) and (ii).

While various example embodiments have been described above, it should be understood that they have been presented by way of example, and not limitation. It will be apparent to persons skilled in the relevant art(s) that various changes in form and detail can be made therein. Thus, the present invention should not be limited by any of the above-described example embodiments but should be defined only in accordance with the following claims and their equivalents.

What is claimed is:

1. A method for determining medical codes using a multi-tier plurality of models, the method comprising:
applying a first-level medical procedure model to data to determine a medical procedure code category;
based on the medical procedure code category, selecting a second-level medical procedure model from a plurality of second-level medical procedure models;
applying the selected second-level medical procedure model to the data to determine a medical procedure code;
applying a first-level medical classification model to the data to determine a medical classification code category;
based on the medical classification code category, selecting a second-level medical classification model from a plurality of second-level medical classification models; and
applying the selected second-level medical classification model to the data to determine a medical classification code.

2. The method of claim 1, wherein the medical procedure code is a Current Procedural Terminology (CPT) code, and the medical classification code is an International Classification of Diseases, 10th Revision (ICD-10) code.

3. The method of claim 1, wherein the data is extracted from a medical document by performing any one of (i) optical character recognition (OCR); (ii) natural language processing (NLP); or (iii) a combination of (i) and (ii).

4. The method of claim 1, wherein the medical procedure code category and the medical classification code category are associated with any one of (i) an anatomical region; (ii) a medical procedure; or (iii) a combination of (i) and (ii).

5. The method of claim 1, further comprising:
assigning a confidence score to (i) the medical procedure code; (ii) the medical classification code; or (iii) both (i) and (ii); and
outputting the confidence score.

6. The method of claim 1, further comprising creating a pair with the medical procedure code and the medical classification code.

7. The method of claim 1, wherein the multi-tier plurality of models are pre-trained models that are fine-tuned using supervised learning to classify one or more of (i) medical procedure codes; (ii) medical classification codes; or (iii) both (i) and (ii).

8. A method for automatically determining medical codes, comprising:
processing and extracting data from a medical document, including extracting the data from the medical document by performing any one of (i) optical character recognition (OCR); (ii) natural language processing (NLP); (iii) identifying one or more relevant sections of the medical document; or (iv) any combination of (i)-(iii);
determining one or more medical codes for the medical document using a multi-tier plurality of models;
assigning a confidence score to the one or more medical codes; and
generating a medical coding report comprising the one or more medical codes and the confidence score.

9. The method of claim 8, wherein determining the one or more medical codes for the medical document using the multi-tier plurality of models comprises:
applying a first-level medical procedure model to the data to determine a medical code category;
based on the medical code category, selecting a second-level medical model from a plurality of second-level medical models; and
applying the selected second-level medical model to the data to determine the one or more medical codes.

10. The method of claim 9, wherein the medical code category is associated with any one of (iv) an anatomical region; (v) a medical procedure; or (vi) a combination of (iv) and (v).

11. The method of claim 8, wherein the one or more medical codes comprises a medical procedure code and a medical classification code.

12. The method of claim 11, further comprising creating a pair with the medical procedure code and the medical classification code.

13. The method of claim 11, wherein determining the one or more medical codes for the medical document using the multi-tier plurality of models comprises:
applying a first-level medical procedure model to the data to determine a medical procedure code category;
based on the medical procedure code category, selecting a second-level medical procedure model from a plurality of second-level medical procedure models;
applying the selected second-level medical procedure model to the data to determine the medical procedure code;
applying a first-level medical classification model to the data to determine a medical classification code category;
based on the medical classification code category, selecting a second-level medical classification model from a plurality of second-level medical classification models; and
applying the selected second-level medical classification model to the data to determine the medical classification code.

14. The method of claim 8, wherein assigning the confidence score is based on a likelihood the multi-tier plurality of models determined the one or more medical codes correctly.

15. The method of claim 8, further comprising:
determining the confidence score is below a confidence threshold; and
in response to determining the confidence score is below the confidence threshold, sending the medical coding report to a review system.

16. The method of claim 8, wherein determining the one or more medical codes for the medical document using the multi-tier plurality of models comprises:
applying a first-level medical procedure model to the data to determine a medical code category;
based on the medical code category, applying one or more intermediate-level medical models from a plurality of intermediate-level medical models to the data to narrow down candidate medical codes; and
applying a final-level medical model to the data to determine the one or more medical codes.

17. A system comprising:
a memory storage; and
a processing unit coupled to the memory storage, wherein the processing unit is operative to:
process and extract data from a medical document;
determine one or more medical codes for the medical document using a multi-tier plurality of models, comprising to:
apply a first-level medical model to the data to determine a medical code category, and
based on the medical code category, select a second-level medical model from a plurality of second-level medical models, and
apply the selected second-level medical model to the data to determine the one or more medical codes; and
generate a medical coding report comprising the one or more medical codes.

18. The system of claim 17, wherein to process and extract the data from the medical document comprises to:
perform any one of (i) optical character recognition (OCR); (ii) natural language processing (NLP); (iii) identify one or more relevant sections of the medical document; or (iv) any combination of (i)-(iii).

19. The system of claim 17, the processing unit being further operative to:
assign a confidence score to the one or more medical codes, wherein the medical coding report further comprises the confidence score.

20. The system of claim 19, the processing unit being further operative to:
determine the confidence score below a confidence threshold; and
in response to determining the confidence score is below the confidence threshold, send the medical coding report to a review system.

21. The system of claim 17, wherein the medical code category is associated with any one of (i) an anatomical region; (ii) a medical procedure; or (iii) a combination of (i) and (ii).
